(19) United States
(12) Patent Application Publication
(10) Pub. No.: US 2024/0156284 A1
(43) Pub. Date: May 16, 2024

(54) BLANKET, IN PARTICULAR A SUPPORT FOR CONTROLLING THE TEMPERATURE OF LIVING BEINGS

(71) Applicant: KOHLBRAT & BUNZ Gesellschaft m.b.H., Radstadt (AT)

(72) Inventor: Michael GRAF, Zederhaus (AT)

(21) Appl. No.: 18/422,783

(22) Filed: Jan. 25, 2024

Related U.S. Application Data
(63) Continuation of application No. PCT/AT22/60266, filed on Jul. 22, 2022.

(30) Foreign Application Priority Data
Jul. 27, 2021 (AT) A 50620/2021

(51) Int. Cl. A47G 9/02; A61F 7/00

(52) U.S. Cl. CPC A47G 9/0215; A47G 9/0223; A61F 7/0097

(57) ABSTRACT

A blanket, in particular a support for controlling the temperature of living beings, includes an upper layer to faces a living being, in particular a patient or injured being; a lower layer opposite the upper layer; a resilient and air-permeable intermediate layer situated between the upper layer and the lower layer; and an air inlet opening, having a connection for supplying air, in particular warm air, into the region of the intermediate layer. An air outlet opening is provided in an edge region of the blanket remote from the air inlet opening, and at least the greater part of the air supplied via the air inlet opening(s) exits the blanket through the air outlet opening(s). The upper layer is fluid-permeable and the lower layer is substantially airtight and substantially fluid-tight, preferably in the form of plastics film or a coated textile sheet.

The present application is a continuation of International Application PCT/AT2022/060266, filed Jul. 22, 2022. Thus, all of the subject matter of International Application PCT/AT2022/060266 is incorporated herein by reference.

### BACKGROUND OF THE INVENTION

The invention relates to a blanket and to a device for stabilizing and/or immobilizing a living being, in particular a patient or injured being, with such a blanket.

Such blankets are in particular used as supports for controlling the temperature of patients or injured beings. However, they can in principle also be placed on the body. Their function is to control the temperature of the body and—as a support—to support it in as pressure-free a manner as possible.

When used for warming, warm air is supplied for example from a heater fan and is blown into the blanket.

Such blankets are used in particular, but not exclusively, in the medical field to prevent heat loss from patients and/or injured beings to be treated.

An example of a heated blanket according to the state of the art is found in DE 10207793 B4. In the design mentioned there, the warm air blown in via the upper layer escapes from the heated blanket itself directly toward the patient lying thereon and warms them by convective warm-air flow.

Especially in the surgical field, such an air guiding direct to the patient can lead to problems with the requirements for high sterility. In addition, it is difficult with the known design to wick away moisture which comes from the body lying thereon in a targeted manner.

### SUMMARY OF THE INVENTION

The object of the invention is to provide a blanket of the type named at the beginning, with which the named disadvantages can be avoided.

In the case of the subject-matter of the invention, the outflow of the air is not (or at least not primarily) effected via the upper layer directly toward the patient, but rather via separate air outlet openings in an edge region of the blanket, which is remote from the connection for the supply of air. The air then flows out of the blanket in a controlled manner via these air outlet openings.

Thus a much more targeted air guiding than in the state of the art is possible. Via the dimensioning of the air outlet openings, the air flow can also be adjusted in a targeted manner such that a desired degree of inflation of the heated blanket is achieved.

In addition, through the air guiding according to the invention via separate air outlet openings, it is possible to discharge moisture coming from the body, which reaches the inside of the blanket from above through the upper layer, and thus to keep the blanket substantially free ultimately from the penetrating moisture over longer periods of time.

Finally, the air outlet openings according to the invention also allow the connection of air discharge pipes or the like, in order to lead the outgoing air away from an operation site or another sensitive site in a targeted manner. It is thus possible to fulfil high sterility requirements.

In addition, at least one air outlet opening is arranged in an outlet valve, the flow through which can be shut off or adjusted. Thus the flow rate of the air flowing through the blanket can be adapted to different intended uses.

### BRIEF DESCRIPTION OF THE DRAWINGS

Further advantages and details of the invention are explained in more detail on the basis of the following description of the figures, in which:

FIG. 1 is a top view of an embodiment of a blanket according to the invention, in particular a heated support,

FIG. 2 is a schematic side view of the blanket according to FIG. 1,

FIG. 3 is a schematic sectional representation according to the section guide A-A in FIG. 1 through the left, upper end region of the blanket according to FIG. 1,

FIG. 4 is the schematic arrangement of the embodiment of a heated support according to the invention in an operating room with external warm air supply and external warm air discharge, and

FIG. 5 shows an embodiment of a device according to the invention for stabilizing and/or immobilizing a living being, in particular a patient or injured being, with a blanket according to the invention and a granulate-filled cushion which can be evacuated.

### DETAILED DESCRIPTION OF THE INVENTION

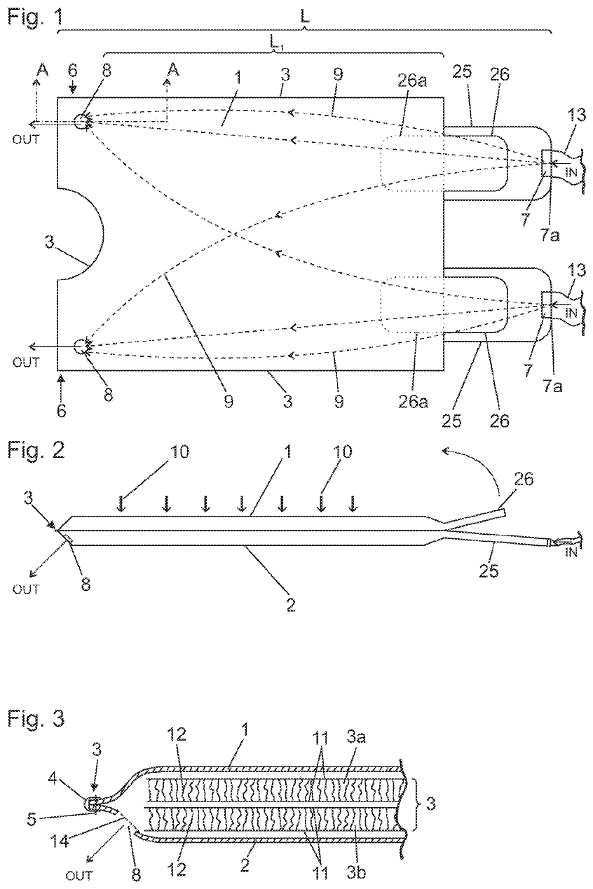

The blanket, in particular support for warming, represented in FIGS. 1 to 3 has an upper layer 1, which faces a living being, in particular a patient or injured being. This can be a human body. In principle, however, the blanket according to the invention is also suitable for animals.

A lower layer 2, which is connected to the upper layer 1 along the edge 6 in an airtight—or substantially airtight—manner, is provided opposite the upper layer 1. This can for example occur in that—as FIG. 3 shows—a beading 4 is provided in the edge region 6, which is sewn to both layers 1 and 2 (see reference number 5).

At least one elastic and air-permeable intermediate layer 3, which is implemented two-layered in the present embodiment (upper layer 3a and lower layer 3b=FIG. 3), is provided between the upper layer 1 and the lower layer 2.

The air intake is effected from the right via a pipe 13 and a connection 7a as well as the actual air inlet openings 7 in FIGS. 1 and 2. These are formed in projecting, flexible tabs 25.

Two further (smaller) tabs 26 can be placed for example on the shoulders of a person in the direction of the swivel arrow of FIG. 2. They then lie as is shown dotted by 26a in FIG. 1.

At least one defined air outlet opening 8 is now provided according to the invention in an edge region 6 of the blanket, which is remote from the air inlet openings 7.

In the present embodiment two air outlet openings 8 are provided, which are arranged in the lower layer 2.

A large part of the air, in particular warm air, flowing in via the air inlet openings 7 flows out via these air outlet openings 8, after it has completely flowed through substantially the entire inside of the heated blanket—thus the intermediate layer lying therein. Some of the air flow lines in the inside are represented by schematic dashed arrow lines with the reference number 9 in FIG. 1.

Such an arrangement of the air outlet openings can be combined in particular with a further variant of the invention, in which it is provided that the upper layer 2 is formed substantially airtight, but preferably moisture-permeable.

In such a variant, moisture coming from the living being lying thereon, for example sweat or blood, but also other moisture penetrating from above can first reach the inside of the blanket, such as is indicated by the double arrows 10 in FIG. 2. The moisture or fluid is then no longer present in a disruptive manner on the upper side on which the body, in particular patient or injured being, is lying.

Through a combination with the air guiding provided according to claim 1, this moisture can now be discharged in a targeted manner from the inside of the blanket via the air outlet openings 8 to the outside, wherein the air is guided through the intermediate layer 3.

The intermediate layer 3 is preferably formed as knitted spacer fabric or woven spacer fabric, which has stiff but yet flexible spacer threads between two textile layers in each case. The textile layers are labeled with the reference number 11, the spacer threads with the reference number 12 (see FIG. 3).

Such knitted or woven spacer fabric has for example polyester threads.

This knitted spacer fabric or woven spacer fabric allows the body lying thereon to be supported pressure-free.

It is simultaneously possible to control the temperature of the body through a targeted warm air supply via the air inlet openings 7. In addition to a warming, in principle a cooling of the body is also possible, if necessary.

The air guiding according to the invention allows targeted, loss-free air streams to be generated. It is thus possible to manage with a smaller heater fan or smaller heating units overall.

In the case of the blanket according to the invention at least 70%, preferably at least 90%, thus a large part of the air stream supplied via the air inlet openings 7 (volume per unit of time) flows out of the air outlet opening(s) 8 according to a preferred embodiment.

In principle, it is also possible that a small part of the air flowing out flows out of the inside of the blanket via other routes, for example as in the state of the art through the upper layer itself.

It is, however, preferably provided that the upper layer is formed substantially airtight, but preferably moisture-permeable. Through the airtight formation of the upper layer, the air can practically only flow out exclusively via the air outlet openings 8 in the case of a lower layer likewise formed airtight and substantially tight seams. This makes a targeted air guiding possible.

The air outlet openings 8 can preferably be formed or arranged in the lower layer and thus do not disrupt the body lying thereon.

The air outlet openings 8 can preferably be formed as circular recesses in the respective layer, such as is indicated by dashed lines (circular) in FIG. 1. Of course, other shapes of the air outlet openings are also possible, for example square or polygonal.

As FIG. 3 shows, at least one air outlet opening has a filter or sieve 14 represented dashed. Thus contamination can be kept inside the blanket and in addition the air escape can be restricted in a targeted manner.

The filter or the sieve 14 can for example consist of a circular, textile sheet, which has relatively large individual openings and which is for example stuck to the inner side of the lower layer 2 around the opening 8.

The lower layer is preferably formed from an airtight and fluid-tight material, for example from a plastic film or a coated textile sheet. Thus moisture which penetrates from above can be prevented from escaping downwards through the blanket.

For a targeted air guiding and a still relatively simple, design structure it is favorable when one to three air inlet openings are provided.

For the same reason, it is also favorable when one to five air outlet openings are provided.

A small number of air inlet openings and outlet openings also allows a simple connection of pipes for the air supply and optionally also for the air discharge.

As FIG. 1 shows, the air inlet openings 7 on one hand and the air outlet openings 8 on the other hand are arranged in opposite edge regions 6 of the blanket.

The blanket has a parallel to the body that can be laid thereon according to its largest longitudinal extent L in the embodiment represented in FIG. 1. A middle region has the length L1. This extends over at least 70% of the entire longitudinal extent L of the blanket. In this middle region with the length L1, preferably no air outlet openings or only much smaller air outlet openings than outside the middle region are provided, thus precisely substantially all of the air flows out in a defined manner defined by the separate, individual air outlet openings.

As already mentioned, the upper layer is favorably formed only slightly air-permeable and preferably actually substantially airtight. It is ensured through such a formation that in a targeted manner substantially the entire or at least a large part of the air stream can flow from the air inlet openings to the separate individual air outlet openings in a defined manner.

Textile sheets are suitable as materials for the upper layer, which form at least one ply of the upper layer. These can preferably consist of plastic threads. Such textile sheets can be easily produced, for example as woven fabric or knitted fabric.

The plastic threads of the textile sheet are preferably polyester threads and particularly preferably with a proportion of over 90%. Carbon fibers can also be used in the textile sheet for stabilization, preferably with a proportion of under 5%. Carbon fibers are very stable and have high tensile strength, but are also expensive, which is why a smaller proportion of carbon fibers is to be preferred. Aside from reasons of stability or strength, the carbon fibers can also be used to prevent electrostatic charges, since they are electroconductive.

The areal weight of the textile sheet is preferably between 90 g/m² and 130 g/m². It has emerged that, with such textile sheets, the relatively low air permeability on one hand and the fluid permeability on the other hand, for example for wicking away blood or sweat, can be simultaneously well achieved.

With the same considerations, it is preferably provided that the fineness of the threads of the textile sheet is under 150 dtex, preferably between 90 dtex and 120 dtex.

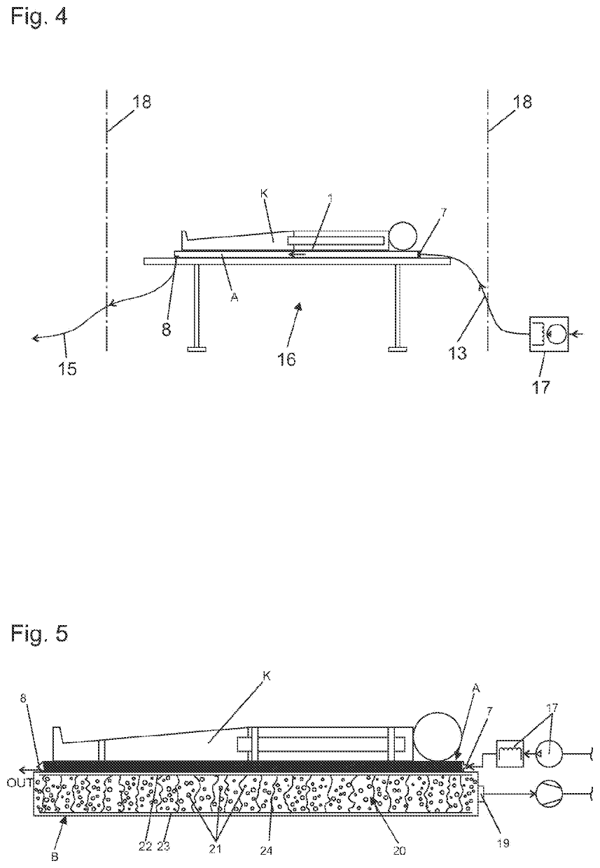

In the case of the embodiment represented in FIG. 4 an operating table 16 for a surgical procedure is schematically represented. On this lies a blanket A according to the invention and lying thereon, a body of a patient and/or injured being.

Warm air is guided via the air inlet openings 7 into the blanket starting from a heater fan 17 via a preferably flexible air supply pipe 13.

This warm air flows through the blanket and out again via the air outlet openings 8. It is preferably provided that no air flows directly toward the body K. Therefore only the upper layer 1 of the blanket is warmed and thus also the body lying thereon.

The air flowing out can be guided to the outside via an air discharge pipe 15.

The vertical dash-dotted lines 18 explain for example an air curtain guided around the operating table or also the wall to a neighboring room, i.e. both the supply air and the discharge air can be supplied and discharged from separate regions.

In the embodiment represented in FIG. 5 the blanket A according to the invention is combined with a cushion B which contains a granulate 21, which is tightly packed and thus stiffens the cushion B after evacuation, in an inner space 20 which can be evacuated via a valve 19.

Such cushions or vacuum mattresses are already known per se. They serve in particular for stabilizing injured beings or patients to be operated on.

Through the combination with the blanket according to the invention, which is formed in particular as a heated support, the body lying thereon can not only be stabilized and immobilized, but also its temperature can be excellently controlled. In addition, a perfect pressure relief of the body lying thereon is provided by the blanket and its elastic interior.

The cushion B is specially formed in the embodiment represented in FIG. 5. It has a granulate 21 and a plug for stabilizing the granulate 21 in the inner space.

The plug is formed by two textile sheets 22, 23 in this embodiment, which are connected to one another via a plurality of threads 24. The granulate 21, for example in the form of small foamed polystyrene balls, is arranged in the edge between the two textile sheets 22, 23 and between the threads 24. The threads 24 function as spacers and taken together with the two textile sheets 22, 23 form knitted spacer fabric or woven spacer fabric.

In contrast to the threads 12 in the decompressed blanket A, the threads 24 in the cushion B do not necessarily have to be dimensionally stable themselves. Their function is mainly to keep the two layers 22, 23 at a constant, maximum distance, which is preferably equal everywhere.

As for the application, the blanket A according to the invention is preferably arranged between the body K and the cushion B.

However, it is also possible that the blanket lies only under the patient or injured being and the layer for stabilization is arranged laterally, for example in the form of two lateral cushions (not represented here) joined to the blanket.

Warm air above room temperature, thus above 20° C., can for example be supplied via a heater fan in the invention. It is in particular also provided to supply air at a higher temperature, for example above body temperature—thus above 36° C.

As already mentioned, a cold air supply is also possible in principle, to cool bodies lying thereon, for example in the case of febrile illnesses. The blanket according to the invention is preferably suitable as a support, in particular a heated support. However, it can in principle also be placed on patients.

1. A blanket, in particular a support for controlling the temperature of living beings, comprising:
an upper layer to face a living being, in particular a patient or injured being,
a lower layer opposite the upper layer,
at least one elastic and air-permeable intermediate layer arranged between the upper layer and the lower layer, and
at least one air inlet opening with a connection for the supply of air, in particular warm air, into the region of the intermediate layer,
wherein at least one air outlet opening is provided in an edge region of the blanket remote from the at least one air inlet opening, wherein at least a large part of the air supplied via the air inlet opening(s) escapes from the blanket through this(these) air outlet opening(s), wherein the upper layer is formed fluid-permeable and in that the lower layer is formed substantially airtight and substantially fluid-tight, preferably as a plastic film or as a coated textile sheet.

2. The blanket according to claim 1, wherein the air inlet opening(s) on one hand and the air outlet opening(s) on the other hand are arranged in opposite edge regions of the blanket.

3. The blanket according to claim 1, wherein at least 70%, preferably at least 90%, of the air stream supplied via the air inlet opening(s) escapes from the air outlet opening(s).

4. The blanket according to claim 1, wherein one to three air inlet openings are provided.

5. The blanket according to claim 1, wherein one to five air outlet openings are provided.

6. The blanket according to claim 1, wherein the upper layer is formed only slightly air-permeable and preferably substantially airtight.

7. The blanket according to claim 1, wherein the upper layer has at least one ply consisting of a textile sheet, preferably consisting of plastic surfaces.

8. The blanket according to claim 7, wherein the plastic surfaces consist of polyester, preferably in a proportion of over 90%, and/or carbon fibers, preferably with a proportion of under 5%.

9. The blanket according to claim 7, wherein the areal weight of the textile sheet is between 90 g/m² and 130 g/m².

10. The blanket according to claim 7, wherein the fineness of the threads of the textile sheet is under 150 dtex, preferably between 90 dtex and 120 dtex.

11. The blanket according to claim 1, wherein substantially the entire air stream supplied via the air inlet opening(s) escapes from the blanket through the air outlet opening(s).

12. The blanket according to claim 1, wherein at least one air outlet opening—preferably all air outlet openings—is (are) formed or arranged in the lower layer.

13. The blanket according to claim 1, wherein the at least one air outlet opening is formed as a preferably circular recess in the lower or upper layer.

14. The blanket according to claim 1, wherein the at least one air outlet opening has a filter or a sieve, whereby contamination can be kept inside the blanket and/or the air escape is restricted.

15. The blanket according to claim 14, wherein the filter or the sieve consists of a net-like textile sheet, which is different from the upper and lower layer.

16. The blanket according to claim 1, wherein at least one air outlet opening is arranged in an outlet valve, the flow through which can be shut off or adjusted.

17. The blanket according to claim 1, wherein the intermediate layer has at least one knitted spacer fabric or woven spacer fabric or the like, in which stiff but yet flexible spacer threads are arranged between two—preferably textile—layers in each case.

18. The blanket according to claim 17, wherein the intermediate layer is formed two- or multilayered, and preferably has two different knitted spacer fabrics or woven spacer fabrics or the like lying loosely one on top of the other.

19. The blanket according to claim 1, wherein a largest longitudinal extent measured parallel to the living being that can be laid thereon, wherein in a middle region of the blanket, the length of which extends over at least 70% of the entire longitudinal extent of the blanket, no air outlet openings or only much smaller air outlet openings than outside this middle region are provided.

20. The blanket according to claim 1, wherein a device for supplying warm air, in particular a heater fan, —preferably via a flexible pipe—can be connected to the at least one detachable connection.

21. The blanket according to claim 1, wherein the air outlet opening(s) have a detachable connection for an air discharge pipe in each case.

22. A device for stabilizing and/or immobilizing a living being, in particular a patient or injured being, comprising the blanket according to claim 1 and at least one cushion, which contains a granulate, which is tightly packed and thus stiffens the cushion after evacuation, in an inner space which can be evacuated via a valve.

23. The device according to claim 22, wherein a granulate and a plug for stabilizing the granulate are arranged in the inner space, wherein the plug has two textile sheets, wherein the two textile sheets are connected to one another by means of a plurality of threads, wherein the granulate is arranged in a space between the two textile sheets and between the threads.

24. The device according to claim 23, wherein the plug is formed in the style of a knitted spacer fabric or a woven spacer fabric.

25. The device according to claim 22, wherein the cushion and the blanket lie one on top of the other, wherein the blanket can be arranged substantially between cushion and patient or injured being.

26. The device according to claim 22, wherein the cushion and the blanket lie laterally next to one another and preferably can be connected to a unit, in which the blanket lies substantially under the patient or injured being and two cushions can be laid laterally against the patient to stabilize them.
